(19) United States
(12) Patent Application Publication
(10) Pub. No.: US 2024/0261311 A1
(43) Pub. Date: Aug. 8, 2024

(54) METHODS OF TREATING OR PREVENTING ALLERGIES AND CHRONIC NASAL CONGESTION

(71) Applicant: Revelation Biosciences, Inc., San Diego, CA (US)

(72) Inventors: James M. Rolke, Tucson, AZ (US); Robin L. Marsden, EI Cajon, CA (US); Shannan J. Lynch, Vista, CA (US); Jackson R. Stephens, San Diego, CA (US); Joseph M. Ninosky, La Jolla, CA (US)

(21) Appl. No.: 18/562,696

(22) PCT Filed: May 26, 2022
(86) PCT No.: PCT/US22/31075

§ 371 Date: Nov. 20, 2023

Related U.S. Application Data
(60) Provisional application No. 63/193,952, filed on May 27, 2021.

(51) Int. Cl. A61K 31/7028; A61K 9/00; A61K 9/107; A61K 9/14; A61K 45/06; A61K 47/10; A61K 47/24

(52) U.S. Cl. CPC A61K 31/7028; A61K 9/0043; A61K 9/107; A61K 9/145; A61K 9/146; A61K 45/06; A61K 47/10; A61K 47/24

(57) ABSTRACT

The present disclosure provides a method of treating or preventing symptoms of allergic rhinitis or chronic nasal congestion in a subject by administering to the subject an MPLA compound.

### CROSS-REFERENCE TO RELATED APPLICATIONS

This application claims the benefit of priority to U.S. Provisional Application No. 63/193,952, filed on May 27, 2021, which is hereby incorporated by reference in its entirety.

### BACKGROUND OF THE INVENTION

Allergies are caused by the body's overreaction to normally well-tolerated proteins called allergens. Allergy symptoms include nasal congestion, sneezing, itchy watery eyes, and rash, each of which may be caused by pollen, grass, weeds, animal dander, or molds. According to the CDC, allergies are currently ranked as the 6th leading chronic disease according to the Asthma and Allergy Foundation of America. Allergic rhinitis or “hayfever” is the most common allergy diagnosis and is a common cause of nasal congestion. 19.2 million adults (7.7% of the general population) were diagnosed with allergic rhinitis in the last 12 months (CDC). In the same time frame, 7.1 million children have been diagnosed with respiratory allergies, and of those, 5.2 million children (7.2% of the population of children age 18 or less) were diagnosed with allergic rhinitis (CDC).

A common symptom of allergic rhinitis, nasal congestion, is also referred to as rhinosinusitis, which is inflammation of the paranasal sinuses and adjacent nasal mucosa. Congestion associated with rhinosinusitis can be acute, with symptoms lasting less than a month, or chronic with symptoms lasting 12 weeks or longer. In clinical practice, rhinosinusitis is one of the most common diagnoses affecting 1 in 6 adults in the Unites States. There are a number of medications currently available for allergies, including antihistamines, decongestants, and steroidal sprays. Nevertheless, many of the currently available treatments are not very effective, or may cause unpleasant side effects, including drowsiness, dry mouth, or increased blood pressure.

Accordingly, there remains a significant need for effective therapies for treating and preventing allergic rhinitis and chronic congestion.

### BRIEF SUMMARY OF THE INVENTION

The present disclosure provides methods of treating or preventing allergic rhinitis or chronic nasal congestion in a subject comprising intranasally administering to the subject an MPLA compound. The MPLA compound may be administered in a dose from about 0.1 mcg to about 200 mcg, about 10 mcg to about 100 mcg, or about 25 mcg to about 75 mcg. In some embodiments, the MPLA compound is administered in a dose of about 50 mcg.

The MPLA compound may be administered to each nasal passage of the subject, for example by dividing the total dose in half an applying one half of the dose to each nasal passage. Alternatively, the full dose of the MPLA compound may be administered to a single nasal passage of the subject.

In particular embodiments, the composition is a colloidal suspension comprising MPLA compound. The colloidal suspension may comprise particles of having a size of about 50 nm to about 1000 nm in diameter or length.

The MPLA compound may advantageously be provided as a composition formulated for nasal administration. The composition may be an aqueous liquid or a powder. When the composition is an aqueous liquid, the MPLA compound may be present in an amount of about 1 mcg/mL to about 1000 mcg/mL, about 20 mcg/mL to about 500 mcg/mL, about 100 mcg/mL to about 300 mcg/mL, about 250 mcg/mL, or about 125 mcg/mL.

Compositions may further comprise an organic solvent, such as a water-miscible organic solvent. Organic solvents that may be useful in the present compositions include an alcohol, glycerin, low molecular weight polyethylene glycol, a poloxamer, or any combination thereof. Alcohols useful in the present formulations include methanol, ethanol, isopropanol, t-butanol, or any combination thereof. Preferably, the alcohol is ethanol.

In certain embodiments, the composition further comprises a sugar. In some embodiments, the sugar is chosen from a monosaccharide, a disaccharide, a trisaccharide, a linear oligosaccharide, a branched oligosaccharide, a cyclic oligosaccharide, a linear polysaccharide, a branched polysaccharide, or any combination thereof.

In some embodiments, the monosaccharide is selected from glucose, dextrose, fructose, galactose, xylose, ribose, and any combination thereof.

In some embodiments, the disaccharide is selected from trehalose, sucrose, maltose, lactose, and any combination thereof.

In some embodiments, the trisaccharide is selected from nigerotriose, maltotriose, melezitose, maltotriulose, raffinose, kestose. and any combination thereof.

In some embodiments, the linear or branched oligosaccharide is selected from nigerotetraose, maltotetraose, lychnose, nystose, sesamose, stachyose.

In some embodiments, the cyclic oligosaccharide is selected from alpha-cyclodextrin, beta-cyclodextrin, or gamma-cyclodextrin.

In some embodiments, the linear or branched polysaccharide is selected from starch, glucan, chitosan, pectin, carboxymethyl cellulose, glycosylaminoglycans, hyaluronic acid, cellulose derivatives, hydroxypropylmethylcellulose (HPMC), dextran, and any combination thereof.

In some preferred embodiments, the sugar comprises trehalose, beta-cyclodextrin, or both.

Compositions according to the present disclosure may also include a fatty acid salt, a fatty acid, a phospholipid, or any combination thereof. Useful phospholipids include phosphatidic acid (PA), phosphatidylcholine (PC), phosphatidylglycerol (PG), phophatidylethanolamine (“PE”), phophatidylinositol (PI), and phosphatidylserine (PS), sphingomyelin (including brain sphingomyelin), lecithin, lysolecithin, lysophosphatidylethanolamine, cerebrosides, diarachidoylphosphatidylcholine (DAPC), didecanoyl-L-alpha-phosphatidylcholine (DDPC), dielaidoylphosphatidylcholine (DEPC), dilauroylphosphatidylcholine (DLPC), dilinoleoylphosphatidylcholine, dimyristoylphosphatidylcholine (DMPC), dioleoylphosphatidylcholine (DOPC), dipalmitoylphosphatidylcholine (DPPC), distearoylphosphatidylcholine (DSPC), 1-palmitoyl-2-oleoyl-phosphatidylcholine (POPC), diarachidoylphosphatidylglycerol (DAPG), didecanoyl-L-alpha-phosphatidylglycerol (DDPG), dielaidoylphosphatidylglycerol (DEPG), dilauroylphosphatidylglycerol (DLPG), dilinoleoylphosphatidylglycerol, dimyristoylphosphatidylglycerol (“DMPG”), dioleoylphosphatidylglycerol (DOPG), dipalmitoylphosphatidylglycerol (“DPPG”), distearoylphosphatidylglycerol (DSPG), 1-palmitoyl-2-oleoyl-phosphatidylglycerol (POPG), diarachidoylphosphatidylethanolamine (DAPE), didecanoyl-L-alpha-phosphatidylethanolamine (DDPE), dielaidoylphosphatidylethanolamine (DEPE), dilauroylphosphatidylethanolamine (DLPE), dilinoleoylphosphatidylethanolamine, dimyristoylphosphatidylethanolamine (DMPE), dioleoylphosphatidylethanolamine (DOPE), dipalmitoylphosphatidylethanolamine (DPPE), distearoylphosphatidylethanolamine (DSPE), 1-palmitoyl-2-oleoyl-phosphatidylethanolamine (POPE), diarachidoylphosphatidylinositol (DAPI), didecanoyl-L-alpha-phosphatidylinositol (DDPI), dielaidoylphosphatidylinositol (DEPI), dilauroylphosphatidylinositol (DLPI), dilinoleoylphosphatidylinositol, dimyristoylphosphatidylinositol (DMPI), dioleoylphosphatidylinositol (DOPI), dipalmitoylphosphatidylinositol (DPPI), distearoylphosphatidylinositol (DSPI), 1-palmitoyl-2-oleoyl-phosphatidylinositol (POPI), diarachidoylphosphatidylserine (DAPS), di decanoyl-L-alpha-phosphatidylserine (DDPS), dielaidoylphosphatidylserine (DEPS), dilauroylphosphatidylserine (DLPS), dilinoleoylphosphatidylserine, dimyristoylphosphatidylserine (DMPS), dioleoylphosphatidylserine (DOPS), dipalmitoylphosphatidylserine (DPPS), distearoylphosphatidylserine (DSPS), 1-palmitoyl-2-oleoyl-phosphatidylserine (POPS), diarachidoyl sphingomyelin, didecanoyl sphingomyelin, dielaidoyl sphingomyelin, dilauroyl sphingomyelin, dilinoleoyl sphingomyelin, dimyristoyl sphingomyelin, sphingomyelin, dioleoyl sphingomyelin, dipalmitoyl sphingomyelin, distearoyl sphingomyelin, 1-palmitoyl-2-oleoyl-sphingomyelin, and any combination thereof. Preferably, the phospholipid is DPPC.

Compositions disclosed herein may further comprises a sugar. Useful sugars include sucrose, glucose, fructose, lactose, maltose, mannose, galactose, trehalose, and combinations thereof.

The compositions in some embodiments further comprise a pH modifier, a pH buffer, a tonicity modifier, a stabilizer, an emulsifier, a preservative, a surfactant, a bulking agent, a flavorant, or any combination thereof.

Composition may further advantageously comprise a mucoadhesive, such as cellulose derivatives, polyacrylates, a starch, chitosan, glycosaminoglycans, hyaluronic acid, and any combination thereof. The mucoadhesive may be present in the composition at about 0.1% to about 10% by weight.

An MPLA compound or composition comprising an MPLA compound may be administered within 14 days, 5 days, or 12 to 72 hours of within 14 days of onset of symptoms of allergic rhinitis or chronic nasal congestion.

Alternatively, the MPLA compound may be administered to the subject within 14 days, 5 days, or 12 to 72 hours of known or suspected exposure of the subject to an allergen.

Administration of the composition may be once every 12 hours, once every 24 hours, once every 48 hours, or once every 72 hours. Administration of the MPLA compound may continue for a treatment course of 1 to 4 weeks, or until the subject until the subject no longer experiences symptoms or is no longer exposed to the allergen.

MPLA compounds useful for administration in the present methods include natural and synthetic MPLA compounds. The MPLA compound may be phosphorylated hexaacyl disaccharide (PHAD), PHAD-504, 3D-PHAD, 3D-(6-acyl) PHAD, or any combination thereof. In certain embodiments, the MPLA compound is PHAD.

Compositions used in the methods of the present invention may be substantially free from a surfactant, a phospholipid, a salt, or a buffer.

Methods of treating or preventing allergic rhinitis or chronic nasal congestion also may further include concurrent administration with at least one additional therapy. The additional therapy may comprise an antihistamines, a decongestants, or a steroidal spray. More particularly, the at least one additional therapy can include the at least one additional therapy comprises diphenhydramine, loratadine, fexofenadine, cetirizine, brompheniramine, desloratadine, azelastine nasal, pseudophedrine, phenylephrine, oxymetazoline, and combinations thereof.

### BRIEF DESCRIPTION OF THE FIGURES

FIG. 1 provides a listing of TRIF-dependent IFN-inducible genes and MYD88/TRIF-dependent proinflammatory cytokines/chemokines.

FIG. 2 shows the structures of various bacterially-derived LPS species.

FIG. 3 is a schematic drawing showing the molecular mechanism of stimulation of interferon production by an exemplary MPLA compound via TLR4 agonization.

FIG. 4 is a schematic illustrating that Type I interferon production blocks activation of Th2 cellular activity at the molecular level, which prevents generation of Th2, reducing or eliminating secretion of IgE, therefore reducing or preventing allergic symptoms (Adapted from Gonzales-van Horn, S. R. and Farrar, J. D. (2015), Interferon at the crossroads of allergy and viral infections. Journal of Leukocyte Biology, 98: 185-194. https://doi.org/10.1189/jlb.3RU0315-099R).

FIG. 5 IP-10 upregulation as a function of dose using representative formulated PHAD. Multiple preparations of PHAD were assessed in an in vitro cell based activity assay using mouse macrophages of the J774 cell line to demonstrate stimulation of TLR4 in response to PHAD as measured by upregulation of IP-10.

FIG. 6 Results of an ad hoc analysis of IP-10 upregulation in response to composition of PHAD contemplated in the current invention in normal healthy human volunteers.

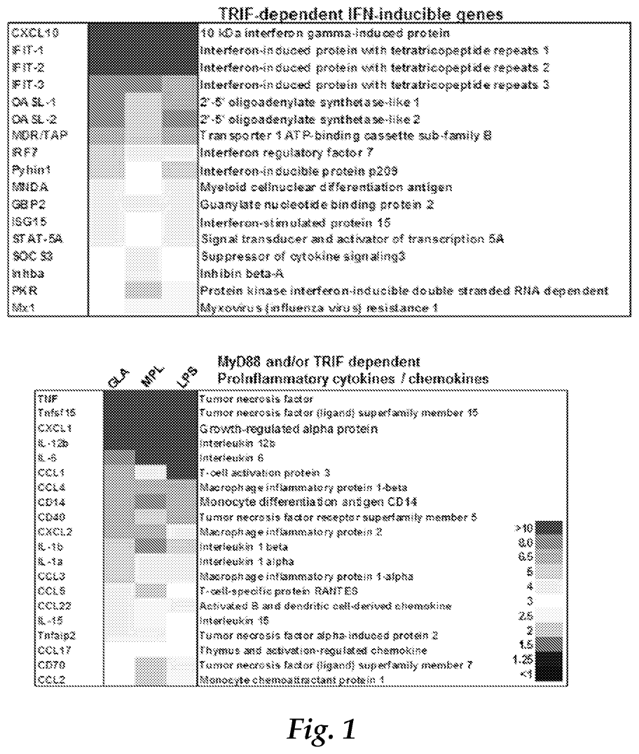

### DETAILED DESCRIPTION OF THE INVENTION

The invention provides a safe and effective method of treating a subject suffering from allergic rhinitis or chronic nasal congestion, or preventing allergic rhinitis or chronic nasal congestion comprising administering to the subject an MPLA compound.

“About” and “approximately” shall generally mean within an acceptable degree of error for the quantity measured given the nature or precision of the measurements. Typically, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value.

### MPLA Compounds

The methods of treatment and prevention of allergies and chronic nasal congestion include administration of an MPLA compound. The MPLA compound may be natural (for example isolated from gram-negative bacterial cell walls) or may be a synthetic MPLA compound.

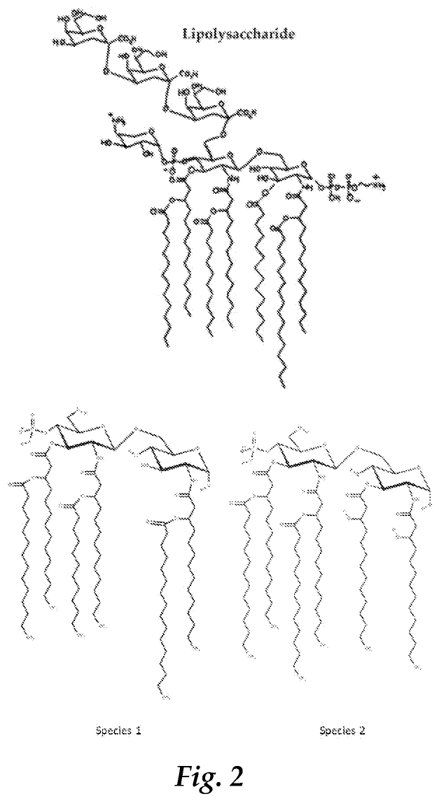

Bacterial MPLA is typically a mixture of several different species. FIG. 2 shows the predominant species of bacterial MPLA. As an example, MPLA may be derived from Salmonella minnesota R595 lipopolysaccharides (LPS). (See FIG. 2.) As will be understood, MPLA may also be derived from other Salmonella species. The bacterial LPS may be processed via sequential acid and alkaline hydrolysis steps to remove polysaccharide side chains, phosphate groups, and to partially remove a portion of the acetyl side groups. The crude MPLA may then be purified. The final MPLA product is a mixture of heptaacyl-, hexaacyl-, and pentaacyl-monophosphorylated glucosamine disaccharide linked ß1->6. Diaacetyl, triaacetyl, and tetraacetyl, if present, are typically considered impurities. The acylated lipids vary and include lauroyl, myristoyl, and palmitoyl. While the relative ratio of each species can vary from batch to batch, the predominant species produced are the hexaacylated disaccharide products.

The major species found in bacterial MPLA have been chemically synthesized and have comparable immunostimulatory properties to the bacterial-derived material. Examples of synthetic MPLA compounds suitable for use in the present invention include phosphorylated hexaacyl disaccharide (PHAD®) (also known as glucopyranosyl lipid A, or GLA), 3D-PHAD (or 3-acyl-PHAD) (also known as monophosphoryl 3-deacyl lipid A), PHAD-504, and 3D-(6-acyl) PHAD (or 3,6-acyl PHAD). Synthetic variations of MPLA that are also suitable and within the scope of the invention include those wherein the fatty acid chain length varies between 10-20 carbons and those wherein the degree of acylation is penta-, hexa-, or hepta-.

PHAD is chemically equivalent to a major component of bacterially-derived MPLA. PHAD is also equivalent to bacterially-derived MPLA in biologic effect. The structure of PHAD is shown below.

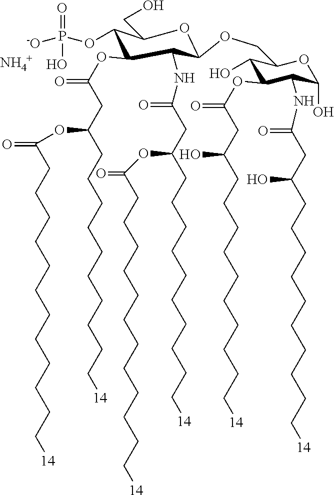

The structure of 3D-PHAD is shown below.

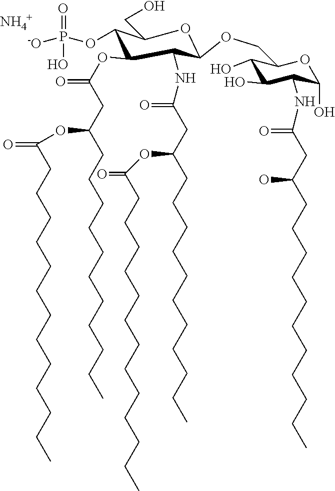

The structure of 3D-(6-acyl) PHAD is shown below.

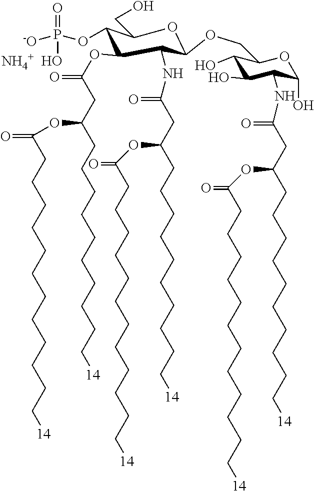

The structure of PHAD-504 is shown below.

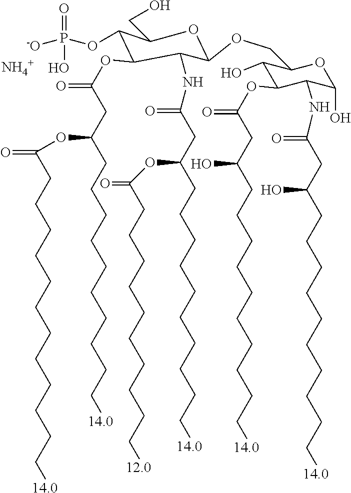

While not being bound by theory, it is believed that MPLA compounds may be able to treat or prevent allergic rhinitis and the associated congestion-causing inflammation by reducing or blocking the signaling and recruitment of cytotoxic immune cells, such as eosinophils, mast cells, and basophils. PHAD binds to Toll-Like Receptor 4 (TLR4), and via downstream cellular signaling, induces upregulation of a number of local cytokines and chemokines, including IP-10 (CXCL10). These early and locally-produced cytokines and chemokines comprise part of the innate immune response, and have the capability to activate the adaptive immune response.

TLR4 stimulation via MPLA binding (TLR4:MPLA) has been shown to preferentially activate the MyD88-independent signaling pathway (TRIF and TRAM signaling pathways) that leads to the synthesis of Type I interferons, the principal T Helper 1 cell (Th1) effector cytokines. Below is a schematic summarizing the TRIF-biased signaling observed in response to MPLA binding to TLR4.

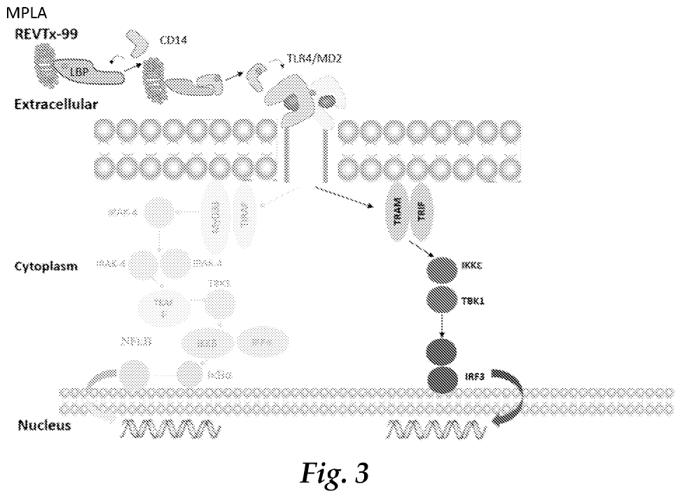

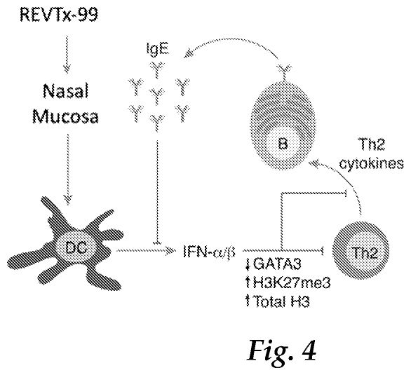

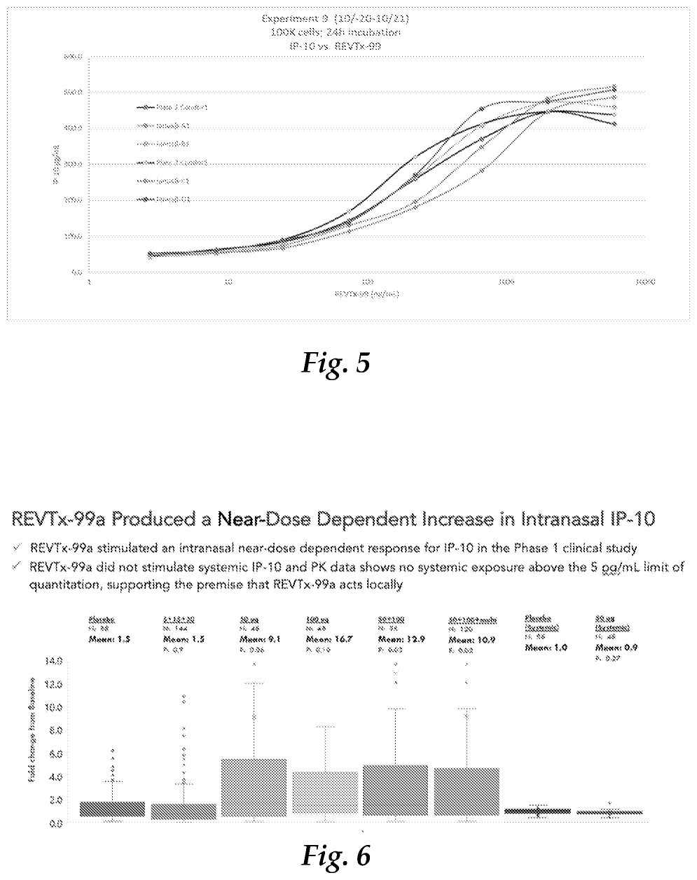

The stimulation of TLR4 in response to PHAD leads to generation of Type I interferons preferentially through the TRIF pathway (FIG. 3). A negative reciprocal feedback loop has been observed to exist between Type I interferon activity and T Helper 2 cell (Th2)-biased cellular activity (Gonzales-van Horn, S. R. and Farrar, J. D. (2015), Interferon at the crossroads of allergy and viral infections. Journal of Leukocyte Biology, 98: 185-194. https://doi.org/10.1189/jlb.3RU0315-099R). This relationship is illustrated in FIG. 4, which details how Type I interferon production blocks activation of Th2 cellular activity at the molecular level (suppression of GATA3 expression, preventing access to transcription mediated expression for H3K27me3 and Total H3), which prevents generation of Th2 cytokines (IL-4, IL-5, and IL-13), reducing or eliminating secretion of IgE, therefore reducing or preventing allergic symptoms.

This induction of Type I interferons reduces or prevents Th2-biased cellular activity (which normally facilitates the allergic response), destabilizing establishment of Th2-biased cellular environment, preventing further allergic response.

TRIF-biased signaling in response to stimulation of MPLA:TLR4 generates IP-10 by different cell types including epithelial cells, macrophages, and dendritic cells. The ability of IP-10 to recruit Th1 cells has been well documented (Sauty, A et al. “The T cell-specific CXC chemokines IP-10, Mig, and I-TAC are expressed by activated human bronchial epithelial cells.” Journal of immunology (Baltimore, Md.: 1950) vol. 162,6 (1999): 3549-58.; Cheng Qian, Huazhang An, Yizhi Yu, Shuxun Liu, Xuetao Cao; TLR agonists induce regulatory dendritic cells to recruit Th1 cells via preferential IP-10 secretion and inhibit Th1 proliferation. Blood 2007; 109 (8): 3308-3315. doi: https://doi.org/10.1182/blood-2006-08-040337). The receptor for IP-10, CXCR3, is present on activated Type I Helper T cells (Staphylococcus aureus Downregulates IP-10 Production and Prevents Th1 Cell Recruitmen; Zhigang Li, Benoît Levast, Joaquín MadrenasThe Journal of Immunology Mar. 1, 2017, 198 (5) 1865-1874; DOI: 10.4049/jimmunol.1601336). Pius Loetscher, Antonio Pellegrino, Jiang-Hong Gong, Ivan Mattioli, Marcel Loetscher, Giuseppe Bardi, Marco Baggiolini, Ian Clark-Lewis, The Ligands of CXC Chemokine Receptor 3, I-TAC, Mig, and IP10, Are Natural Antagonists for CCR3*, Journal of Biological Chemistry, Volume 276, Issue 5, 2001, Pages 2986-2991, ISSN 0021-9258, https://doi.org/10.1074/jbc.M005652200. (https://www.sciencedirect.com/science/article/pii/S002192581846468X)). IP-10 is also capable of binding to CCR3, the native receptor for eotaxin, acting as an antagonist of CCR3-mediated chemotaxis. The CCR3 receptor is present on a number of cells involved in the allergic response, including eosinophils, basophils, and mast cells. IP-10 can directly compete with eotaxin for binding to its native receptor, preventing eotaxin from recruiting immune cells such as eosinophils and basophils, reducing further recruitment of Th2 cells, and attenuating the allergic response. The presence of IP-10, in conjunction with the observed bias toward Th1 cell signaling (a bias away from the allergic Type 2 helper cell population), indicates an anti-allergic role for MPLA.

### Compositions

In some embodiments, the MPLA compound is administered in a composition comprising one or more pharmaceutically acceptable excipients. The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

For example, the MPLA compound can be formulated for intranasal delivery as a dry powder, as an aqueous solution, an aqueous suspension, a colloidal suspension, a water-in-oil emulsion, a micellar formulation, or as a liposomal formulation.

In some embodiments, the MPLA composition comprises a colloid comprising the MPLA compound. While not being bound by theory, it is believed that a colloid enhances the antiviral activity of the MPLA. The colloid may comprise micelles, liposomes, vesicles or particles having a size of about 50 nm to about 1000 nm. The size of micelles, liposomes, vesicles or particles may be measured by various techniques, including dynamic light scattering (DLS), scanning electron Microscopy (SEM), transmission electron microscopy (TEM). Accordingly, in some embodiments, the size of the micelles, liposomes, vesicles or particles is about 50 nm to about 1000 nm as measured by DLS.

“Colloid” as used herein, refers to any liquid or solid composition comprising multimolecular aggregate microstructures having diameters or lengths on the scale of 1 nm to 10 μm. Such microstructures include but are not limited to micelles, liposomes, vesicles, nanoparticles, microparticles, etc. The microstructures may be spherical, oval, oblong, flat, or any other shape.

“Micelles,” as used herein, is an art-recognized term and refers to particles of colloidal dimensions that exist in equilibrium with the molecules or ions in solution from which it is formed. It is an aggregate (or supramolecular assembly) of molecules dispersed in a liquid, forming a colloidal suspension (also known as associated colloidal system). A typical micelle in water forms an aggregate with the hydrophilic “head” regions in contact with surrounding solvent, sequestering the hydrophobic single-tail regions in the micelle center.

“Liposome,” as used herein, is an art-recognized term and refers to a spherical vesicle having at least one lipid bilayer. Liposomes can be prepared by disrupting biological membranes (such as by sonication).

“Vesicle,” as used herein is an art-recognized term and refers to a membranous fluid filled sac surround by a lipid bilayer.

“Nanoparticle,” as used herein, is an art-recognized term and is typically defined as a particle of matter that is between 1 and 100 nanometers (nm) in diameter. The term is sometimes used for larger particles, up to 500 nm.

“Microparticle,” as used herein, is an art-recognized term and is defined to be particles between 1 and 1000 μm in size. The size of micelles, liposomes, vesicles or particles may be measured by various techniques, including dynamic light scattering (DLS), scanning electron Microscopy (SEM), and transmission electron microscopy (TEM). Accordingly, in some embodiments, the size of the micelles, liposomes, vesicles or particles is about 50 nm to about 1000 nm as measured by DLS.

In certain embodiments, the composition is a colloidal suspension comprises micelles. In certain embodiments, the colloidal suspension comprises liposomes. In certain embodiments, the colloidal suspension comprises nanoparticles. In certain embodiments, the colloidal suspension comprises microparticles.

In some embodiments, the composition further comprises an organic solvent such as an alcohol, glycerin, low molecular weight polyethylene glycol, a poloxamer (e.g., poloxamer 407, poloxamer 181), or any combination thereof. In some embodiments, the organic solvent is water miscible. In some embodiments, the organic solvent is an alcohol such as methanol, ethanol, isopropanol, or t-butanol. Preferably, the alcohol is ethanol.

In some embodiments, the compositions further comprises a fatty acid salt, fatty acids, a phospholipid, or any combination thereof.

In some embodiments, at least one of the lipids is a phospholipid or a mixture of phospholipids. Examples of phospholipids include, but are not limited to, phosphatidic acid (PA), phosphatidylcholine (PC), phosphatidylglycerol (PG), phophatidylethanolamine (PE), phophatidylinositol (PI), and phosphatidylserine (PS), sphingomyelin (including brain sphingomyelin), lecithin, lysolecithin, lysophosphatidylethanolamine, cerebrosides, diarachidoylphosphatidylcholine (DAPC), didecanoyl-L-alpha-phosphatidylcholine (DDPC), dielaidoylphosphatidylcholine (DEPC), dilauroylphosphatidylcholine (DLPC), dilinoleoylphosphatidylcholine, dimyristoylphosphatidylcholine (DMPC), dioleoylphosphatidylcholine (DOPC), dipalmitoylphosphatidylcholine (DPPC), distearoylphosphatidylcholine (DSPC), 1-palmitoyl-2-oleoyl-phosphatidylcholine (POPC), diarachidoylphosphatidylglycerol (DAPG), didecanoyl-L-alpha-phosphatidylglycerol (DDPG), dielaidoylphosphatidylglycerol (DEPG), dilauroylphosphatidylglycerol (DLPG), dilinoleoylphosphatidylglycerol, dimyristoylphosphatidylglycerol (DMPG), dioleoylphosphatidylglycerol (DOPG), dipalmitoylphosphatidylglycerol (DPPG), distearoylphosphatidylglycerol (DSPG), 1-palmitoyl-2-oleoyl-phosphatidylglycerol (POPG), diarachidoylphosphatidylethanolamine (DAPE), didecanoyl-L-alpha-phosphatidylethanolamine (DDPE), dielaidoylphosphatidylethanolamine (DEPE), dilauroylphosphatidylethanolamine (DLPE), dilinoleoylphosphatidylethanolamine, dimyristoylphosphatidylethanolamine (DMPE), dioleoylphosphatidylethanolamine (DOPE), dipalmitoylphosphatidylethanolamine (DPPE), distearoylphosphatidylethanolamine (DSPE), 1-palmitoyl-2-oleoyl-phosphatidylethanolamine (POPE), diarachidoylphosphatidylinositol (DAPI), didecanoyl-L-alpha-phosphatidylinositol (DDPI), dielaidoylphosphatidylinositol (DEPI), dilauroylphosphatidylinositol (DLPI), dilinoleoylphosphatidylinositol, dimyristoylphosphatidylinositol (DMPI), dioleoylphosphatidylinositol (DOPI), dipalmitoylphosphatidylinositol (DPPI), distearoylphosphatidylinositol (DSPI), 1-palmitoyl-2-oleoyl-phosphatidylinositol (POPI), diarachidoylphosphatidylserine (DAPS), didecanoyl-L-alpha-phosphatidylserine (DDPS), dielaidoylphosphatidylserine (DEPS), dilauroylphosphatidylserine (DLPS), dilinoleoylphosphatidylserine, dimyristoylphosphatidylserine (DMPS), dioleoylphosphatidylserine (DOPS), dipalmitoylphosphatidylserine (DPPS), distearoylphosphatidylserine (DSPS), 1-palmitoyl-2-oleoyl-phosphatidylserine (POPS), diarachidoyl sphingomyelin, didecanoyl sphingomyelin, dielaidoyl sphingomyelin, dilauroyl sphingomyelin, dilinoleoyl sphingomyelin, dimyristoyl sphingomyelin, sphingomyelin, dioleoyl sphingomyelin, dipalmitoyl sphingomyelin, distearoyl sphingomyelin, 1-palmitoyl-2-oleoyl-sphingomyelin, and any combination thereof.

In one embodiment, the phospholipid is DPPC, DOPC, cholesterol, or a mixture thereof.

In certain embodiments, compositions comprising MPLA may contain pH modifiers, pH buffers, tonicity modifiers, stabilizers, preservatives, detergents, flavorants, bulking agents, an emulsifier (such as squalene) or secondary immunostimulatory agents. In some embodiments, the composition is a powder comprising a bulking agent.

Secondary immunostimulatory agents include, e.g., gonadocorticoids, deoxycholic acid, vitamin D, and beta-glucans. Suitable buffers include sodium chloride-based or potassium chloride-based solutions such as phosphate buffered saline, potassium buffered saline, or borate buffered saline. In some embodiments, the buffer may contain salts, detergents, or carbohydrates which preserve the MPLA upon drying and aid in resolubilizing the MPLA upon encounter with a liquid. Suitable carbohydrates include trehalose, sucrose, glucose, and mannose.

In some embodiments, the composition further comprises a mucoadhesive. Mucoadhesives include glycosaminoglycans (GAGS), including chondroitin sulfate, chitosan, hyaluronic acid, cellulose derivatives, polyacrylates, and a starch.

In some embodiments, the mucoadhesive is present in the composition in an amount ranging from about 0.1 to about 10% by weight about 0.1 to about 2% by weight, or about 1% by weight.

In some embodiments, the composition further comprises a sugar. Examples of sugars that may be used in the methods provided herein include, but are not limited to, sucrose, glucose, fructose, lactose, maltose, mannose, galactose, trehalose, and combinations thereof.

In some embodiments, the composition is an aqueous liquid. In such embodiments, the concentration of the MPLA compound in the composition may be about 1 mcg/mL to about 1000 mcg/mL, about 20 mcg/mL to about 500 mcg/mL, about 100 mcg/mL to about 300 mcg/mL, or about 250 mcg/mL.

In certain embodiments, the formulation may contain ionic or nonionic surfactants. Suitable surfactants include dodecyltrimethylammonium bromide (DTAB), n-dodecyl octaethylene oxide (C12E8), n-dodecyl tetraethylene oxide (C12E4) and dioctanoyl phosphatidylcholine (C8-lecithin), polyoxyl 35 castor oil, cremophor EL (CrEL), octaethylene glycol monododecyl ether (C12E8), hexadecyltrimethylammonium bromide (CTAB), polypropylene oxide (PPO), polyethylene oxide (PEO), PEO-poly(D,L-lactic acid-co-caprolactone) (PEO-PDLLA) and sodium dodecyl sulfate (SDS) and any combination thereof.

In some embodiments, the MPLA is formulated at a pH between 4 and 9. In preferred embodiments, the pH is between 5 and 8 and most preferably between 6.5 and 7.5.

In certain embodiments, the formulations may be free of or substantially free of phospholipids, surfactants, salt (e.g., NaCl), and/or buffers. Substantially free means that the substance in question makes up less than 0.5%, less than 0.25%, less than 0.1%, less than 0.05%, less than 0.01%, or less than 0.005% by weight of the composition.

### Dosing and Administration

In accordance with the invention, the MPLA compound can be administered in therapeutically effective amounts to treat or prevent allergic rhinitis or chronic nasal congestion in a subject.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof). Treating allergic rhinitis or chronic nasal congestion may include: alleviation or elimination of symptoms such runny nose, sneezing, itchy water eyes, cough, fatigue, headache, sore throat, or congestion.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats). In some embodiments, the subject is a human.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated.

While not being bound by theory, it is believed that MPLA engages the innate immune system to elicit endogenous cytokines and chemokines including Type I interferons (see FIG. 3), which avoids the risk of developing immunogenicity to endogenous cytokine or chemokine counterparts.

In certain embodiments, MPLA is administered in a total dose between 0.1 to 200 micrograms. The total dose given may be administered intranasally, and may be divided in equal or unequal parts between both nostrils. In preferred embodiments, the total dose administered is between 25 and 75 micrograms. In particularly preferred embodiments, the total dose is about 50 micrograms.

The MPLA compound can be administered systemically or directly to the mucosa (e.g., nasal and/or pulmonary mucosa).

Methods of systemic delivery include those methods known in the art that provide delivery of the active molecule (e.g. the drug) to the circulatory system with distribution throughout the body. Systemic delivery methods include intramuscular, intravenous, subcutaneous, intraperitoneal, and oral. As will be understood, any method of systemic delivery is suitable for use with the invention. Particularly suitable methods of systemic delivery include intramuscular and intravenous delivery.

Methods for mucosal delivery include those methods known in the art that provide delivery of the composition to mucous membranes. Mucosal delivery methods include intranasal, intrabuccal, and oral. In some embodiments, the administration is intranasal.

In these embodiments, the MPLA compound may be formulated to be delivered to the nasal passages or nasal vestibule of the subject as droplets, an aerosol, micelles, lipid or liquid nanospheres, liposomes, lipid or liquid microspheres, a solution spray, or a powder. The composition can be administered by direct application to the nasal passages, or may be atomized or nebulized for inhalation through the nose or mouth.

In some embodiments, the method comprises administering a nasal spray, medicated nasal swab, medicated wipe, nasal drops, or aerosol to the subject's nasal passages or nasal vestibule.

In some embodiments, the compositions of present invention can be delivered using a small needle-free nasal spray device, which can allow (self) administration with little or no prior training to deliver a desired dose. The apparatus can comprise a reservoir containing a quantity of the composition. The apparatus may comprise a pump spray for delivering one or more metered doses to the nasal cavity of a subject. The device may advantageously be single dose use or multi-dose use. It further may be designed to administer the intended dose with multiple sprays, e.g., two sprays, e.g., one in each nostril, or as a single spray, e.g., in one nostril, or to vary the dose in accordance with the body weight or maturity of the patient. In some embodiments, nasal drops may be prepacked in pouches or ampoules that may be opened immediate prior to use and squeezed or squirted into the nasal passages.

In some embodiments, the MPLA compound is administered to the subject within 14 days of symptom onset of allergic rhinitis or chronic nasal congestion, preferably at a time when the symptoms are considered to be mild to moderate, e.g., as perceived by the patient or evaluated by the treating physician. Mild to moderate symptoms include, e.g., runny nose, sneezing, itchy water eyes, cough, fatigue, rash, headache, sore throat, or congestion. Symptoms that are generally not considered minor to moderate include, e.g., trouble breathing, persistent pain or pressure in chest, confusion, inability to wake or stay awake, and bluish lips or face. In further embodiments, the MPLA compound is administered to the subject within 13, 12, 11, 10, 9, 8, 7, 6, 5, 4, or 3 days of mild symptom onset. In other embodiments, the MPLA compound is administered within 12 to 72 hours of symptom onset.

In some embodiments, the MPLA compound is administered prior to or during exposure to an allergen or other event that triggers nasal congestion. In some embodiments, administration is about 1 to 72 hours prior to exposure to an allergen. In other embodiments, the administration is within 13, 12, 11, 10, 9, 8, 7, 6, 5, 4, or 3 days of exposure to an allergen. In other embodiments, the administration is within 12 to 72 hours of exposure. Administration of an MPLA compound prior to or during exposure to an allergen may prevent, delay onset of, and/or may reduce the severity of symptoms.

In certain embodiments, after the first dose is administered, the MPLA compound is further administered according to a dosing schedule. Exemplary dosing schedules include once every 12 hours, once every 24 hours, once every 48 hours, once every 72 hours and once every week, until the subject is asymptomatic or until exposure to allergens is reduced to a tolerable level or eliminated altogether. In some embodiments, the dosing schedule is continued for about 1 week to about 4 weeks. Dosing schedules may be varied in some embodiments. For example, dosing may be every 12 hours for a period of days, followed by every 24 hours, 48 hours, or 72 hours. Variations in the dosing schedule may depend on the severity and persistence of symptoms of allergic rhinitis or chronic nasal congestion.

In some embodiments, intranasal administration is carried out concurrently with administration of an additional therapy for allergic rhinitis or chronic nasal congestion. As used herein, “concurrent administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic agents. Additional therapies include antihistamines, decongestants, and steroidal sprays. Antihistamines include Benadryl (diphenhydramine), Claritin (loratadine), Allegra (fexofenadine), and Zyrtec (cetirizine), and nasal sprays such as Nasahist B (brompheniramine), Clarinex (desloratadine) and Astelin (azelastine nasal). Decongestants available for treatment include Sudafed (pseudophedrine), Neo-Synephrine (phenylephrine), and Afrin (oxymetazoline). Steroidal sprays include fluticasone.

### EXAMPLES

### Example 1: Formulation of PHAD for Intranasal Administration

An exemplary formulation containing PHAD for intranasal administration is set forth in Table 1 below.

TABLE 1

| Material | Amount | |
|---|---|---|
| PHAD | 1 | mg |
| DPPC | 0.84 | mg |
| Water for irrigation | 3.6 | mL |
| Ethanol (absolute) | 0.4 | mL |

PHAD = Phosphorylated hexaacyl disaccharide, DPPC = dipalmitoylphosphatidyl choline.

Another exemplary formulation, which does not include DPPC, is described in Table 2.

| Ingredient | Amount | | Purpose |
|---|---|---|---|
| PHAD | 250 | ug/mL* | Active ingredient |
| Ethanol | 5% | v/v | Diluent used to pre-dissolve PHAD |
| | | | prior to dilution with water |
| Water for irrigation | 95% | v/v | Diluent |

The formulation may be prepared by the following steps: 1 mg of PHAD was wetted for 1 minute in 0.4 mL of 95% ethanol then sonicated for 15 minutes at 40° C. until a clear solution is formed. The solution was removed from the sonication bath and QS to 8 mL with water, resulting in a homogeneous formulation of PHAD micelles 150 nm or smaller in size. The formulation was either used as liquid or lyophilized.

### Example 2: Safety and Tolerability of an Exemplary Formulation Containing PHAD

An exemplary formulation of PHAD as described in Table 2 was evaluated for safety. In part 1, treatment was administered as a single dose of placebo or the study drug (a composition comprising PHAD® as described in Table 2. at 5, 15, 30, 50, and 100 μg. Monitoring of adverse events was conducted throughout the study starting with Day 1, after the first administration of study drug, through the End of Study visit (Day 8). The most frequently reported event was blood on the nasal swab during sample collection. Other events considered related were rhinorrhea, nasal stinging/burning/irritation, lethargy, nasal congestion, lethargy, or musculoskeletal pain, all reported as mild.

No participants experienced a serious adverse event or an adverse event that resulted in study discontinuation and there were no clinically significant laboratory, vital signs, ECG, or physical examination findings. Dose levels are summarized in Table 3.

In part 2 of this study, participants will be administered the study drug at 100 μg per day for five days. The dosing schedule of the study drug is shown in Table 3. Concentrations of PHAD® are 2.5, 12.5, 37.5, 75, 125 and 250 mg/μl.

TABLE 3
Dosing Schedule of Study Drug
Part 1, Cohorts 1-5

| | | | 5% v/v | | | |
|---|---|---|---|---|---|---|
| | | | ethanol | | | |
| | | | in sterile | | | Total |
| | | Stock | water for | mL | | dose |
| | PHAD ® | Vol. | irrigation | per | PHAD ® | PHAD ® |
| Cohort | (μg/mL) | (mL) | (mL) | nostril | (μg/nostril) | (μg) |
| −1 | 2.5 | 0.1 | 4.9 | 0.2 | 0.5 | 1 |
| 1 | 12.5 | 0.5 | 4.5 | 0.2 | 2.5 | 5 |
| 2 | 37.5 | 1.5 | 3.5 | 0.2 | 7.5 | 15 |
| 3 | 75 | 3 | 2 | 0.2 | 15 | 30 |
| 4 | 125 | NA | NA | 0.2 | 25 | 50 |
| 5 | 250 | NA | NA | 0.2 | 50 | 100 |

Part 2, Cohort 1

| | | | 5% v/v | | | |
|---|---|---|---|---|---|---|
| | | | ethanol | | | |
| | | | in sterile | | | Total |
| | | Stock | water for | mL | | dose |
| | PHAD ® | Vol. | irrigation | per | PHAD | PHAD ® |
| Cohort | (μg/mL) | (mL) | (mL | nostril | (μg/nostril | (μg) |
| 1 | 250 | NA | NA | 0.2 | 50 | 100 |

### Example 4

### Preparation of PHAD Micelles in 5% Ethanol.

1 mg of PHAD was wetted for 1 minute in 0.4 mL of 95% ethanol then sonicated for 15 minutes at 40° C. until a clear solution is formed. The solution was removed from the sonication bath and QS to 8 mL with water, resulting in a homogeneous formulation of PHAD micelles 150 nm or smaller in size. The formulation was either used as liquid or lyophilized.

### Example 5

### Preparation of PHAD Powder.

6 mg of PHAD was wetted and dissolved in 3 mL of 95% ethanol at 40° C. and sonicated for 20 minutes at 40° C. until a clear solution of 2 mg/mL PHAD was obtained. 17 mL of water at 40° ° C. was then added and sonicated to fully mix the bulk solution, resulting in a homogeneous formulation of PHAD micelles around 150 nm or smaller in size. 147 mg of HP-β-Cyclodextrin and 147 mg of Trehalose dihydrate were added to the solution under mixing. This solution was then spray dried to obtain a final powder at 2% w/w/w PHAD:HP-β-Cyclodextrin:Trehalose. This particular composition of PHAD was readily soluble in water at concentrations up to 8 mg/mL.

### Example 6

Formulations as prepared in Examples 1 and 2 show robust upregulation of IP-10, as a result of TLR4 stimulation in vitro in mouse macrophages (FIG. 2). IP-10 is an important cytokine associated with stimulation of the TRIF pathway. Evidence of IP-10 upregulation is confirmation that PHAD preparations as noted in this application are capable of selectively stimulating the TRIF pathway, which is further confirmation of the amelioration or reduction of activity mediated through the MyD88 signaling pathway, which supports the amelioration of pro-inflammatory cytokine production for MPLA as a treatment for inflammatory conditions.

### Example 7

Formulations as prepared in Examples 1 show a dose dependent upregulation of IP-10 when administered intranasally to healthy human volunteers (FIG. 6). Presented is a box and whisker plot of the fold-change values for all data and time-points including the mean fold-change and p-value calculated using a t-test comparing placebo to each data set presented.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations

We claim:

1. A method of treating or preventing allergic rhinitis or chronic nasal congestion in a subject, comprising intranasally administering to the subject a monophosphoryl lipid A (MPLA) compound.

2. The method of claim 1, wherein the MPLA is administered in a dose from about 0.1 mcg to about 200 mcg.

3. The method of claim 2, wherein the MPLA is administered in a dose from about 10 mcg to about 100 mcg.

4. The method of claim 3, wherein the MPLA is administered in a dose from about 25 mcg to about 75 mcg.

5. The method of claim 4, wherein the MPLA is administered in a dose of about 50 mcg.

6. The method of any preceding claim, wherein half of the dose of MPLA is administered to each nasal passage of the subject.

7. The method of any one of claims 1 to 5, wherein the entire dose of MPLA is administered to a single nasal passage of the subject.

8. The method of any preceding claim, wherein the MPLA is provided in a composition.

9. The method of claim 8, wherein the composition is a colloidal formulation comprising the MPLA compound.

10. The method of claim 9, wherein the colloidal formulation comprises particles having a size of about 50 nm to about 1000 nm in diameter.

11. The method of any one of claims 8 to 10, wherein the composition comprises an aqueous liquid.

12. The method of claim 11, wherein the MPLA compound is present in the composition at a concentration from about 1 mcg/mL to about 1000 mcg/mL.

13. The method of claim 12, wherein the MPLA compound is present in the composition at a concentration ranging from about 20 mcg/mL to about 500 mcg/mL.

14. The method of claim 13, wherein the MPLA compound is present in the composition at a concentration ranging from about 100 mcg/mL to about 300 mcg/mL.

15. The method of claim 14, wherein the MPLA compound is present in the composition at a concentration of about 250 mcg/mL.

16. The method of claim 14, wherein the MPLA compound is present in the composition at a concentration of about 125 mcg/mL.

17. The method of any one of claims 8 to 16, wherein the composition further comprises an organic solvent.

18. The method of claim 17, wherein the organic solvent comprises an alcohol, glycerin, low molecular weight polyethylene glycol, a poloxamer, or any combination thereof.

19. The method of claim 18, wherein the alcohol comprises methanol, ethanol, isopropanol, t-butanol, or any combination thereof.

20. The method of claim 19, wherein the organic solvent comprises ethanol.

21. The method of any one of claims 8 to 10, wherein the composition is a powder.

22. The method of any preceding claim, wherein the composition further comprises a fatty acid salt, a fatty acid, a phospholipid, or any combination thereof.

23. The method of claim 22, wherein the phospholipid is selected from phosphatidic acid (PA), phosphatidylcholine (PC), phosphatidylglycerol (PG), phophatidylethanolamine (“PE”), phophatidylinositol (PI), and phosphatidylserine (PS), sphingomyelin (including brain sphingomyelin), lecithin, lysolecithin, lysophosphatidylethanolamine, cerebrosides, diarachidoylphosphatidylcholine (DAPC), didecanoyl-L-alpha-phosphatidylcholine (DDPC), dielaidoylphosphatidylcholine (DEPC), dilauroylphosphatidylcholine (DLPC), dilinoleoylphosphatidylcholine, dimyristoylphosphatidylcholine (DMPC), dioleoylphosphatidylcholine (DOPC), dipalmitoylphosphatidylcholine (DPPC), distearoylphosphatidylcholine (DSPC), 1-palmitoyl-2-oleoyl-phosphatidylcholine (POPC), diarachidoylphosphatidylglycerol (DAPG), didecanoyl-L-alpha-phosphatidylglycerol (DDPG), dielaidoylphosphatidylglycerol (DEPG), dilauroylphosphatidylglycerol (DLPG), dilinoleoylphosphatidylglycerol, dimyristoylphosphatidylglycerol (“DMPG”), dioleoylphosphatidylglycerol (DOPG), dipalmitoylphosphatidylglycerol (“DPPG”), distearoylphosphatidylglycerol (DSPG), 1-palmitoyl-2-oleoyl-phosphatidylglycerol (POPG), diarachidoylphosphatidylethanolamine (DAPE), didecanoyl-L-alpha-phosphatidylethanolamine (DDPE), dielaidoylphosphatidylethanolamine (DEPE), dilauroylphosphatidylethanolamine (DLPE), dilinoleoylphosphatidylethanolamine, dimyristoylphosphatidylethanolamine (DMPE), dioleoylphosphatidylethanolamine (DOPE), dipalmitoylphosphatidylethanolamine (DPPE), distearoylphosphatidylethanolamine (DSPE), 1-palmitoyl-2-oleoyl-phosphatidylethanolamine (POPE), diarachidoylphosphatidylinositol (DAPI), didecanoyl-L-alpha-phosphatidylinositol (DDPI), dielaidoylphosphatidylinositol (DEPI), dilauroylphosphatidylinositol (DLPI), dilinoleoylphosphatidylinositol, dimyristoylphosphatidylinositol (DMPI), dioleoylphosphatidylinositol (DOPI), dipalmitoylphosphatidylinositol (DPPI), distearoylphosphatidylinositol (DSPI), 1-palmitoyl-2-oleoyl-phosphatidylinositol (POPI), diarachidoylphosphatidylserine (DAPS), di decanoyl-L-alpha-phosphatidylserine (DDPS), dielaidoylphosphatidylserine (DEPS), dilauroylphosphatidylserine (DLPS), dilinoleoylphosphatidylserine, dimyristoylphosphatidylserine (DMPS), dioleoylphosphatidylserine (DOPS), dipalmitoylphosphatidylserine (DPPS), distearoylphosphatidylserine (DSPS), 1-palmitoyl-2-oleoyl-phosphatidylserine (POPS), diarachidoyl sphingomyelin, didecanoyl sphingomyelin, dielaidoyl sphingomyelin, dilauroyl sphingomyelin, dilinoleoyl sphingomyelin, dimyristoyl sphingomyelin, sphingomyelin, dioleoyl sphingomyelin, dipalmitoyl sphingomyelin, distearoyl sphingomyelin, 1-palmitoyl-2-oleoyl-sphingomyelin, and any combination thereof.

24. The method of claim 24, wherein the phospholipid is DPPC.

25. The method of any preceding claim, wherein the composition further comprises a sugar.

26. The pharmaceutical composition of claim 25, wherein the sugar is chosen from a monosaccharide, a disaccharide, a trisaccharide, a linear oligosaccharide, a branched oligosaccharide, a cyclic oligosaccharide, a linear polysaccharide, a branched polysaccharide, or any combination thereof.

27. The pharmaceutical composition of claim 26, wherein the monosaccharide is selected from glucose, dextrose, fructose, galactose, xylose, ribose, and any combination thereof.

28. The pharmaceutical composition of claim 26 or 27, wherein the disaccharide is selected from trehalose, sucrose, maltose, lactose, and any combination thereof.

29. The pharmaceutical composition of any one of claims 26-28, wherein the trisaccharide is selected from nigerotriose, maltotriose, melezitose, maltotriulose, raffinose, kestose. and any combination thereof.

30. The pharmaceutical composition of any one of claims 26-29, wherein the linear or branched oligosaccharide is selected from nigerotetraose, maltotetraose, lychnose, nystose, sesamose, stachyose.

31. The pharmaceutical composition of any one of claims 26-30, wherein the cyclic oligosaccharide is selected from alpha-cyclodextrin, beta-cyclodextrin, or gamma-cyclodextrin.

32. The pharmaceutical composition of any one of claims 26-31, wherein the linear or branched polysaccharide is selected from starch, glucan, chitosan, pectin, carboxymethyl cellulose, glycosylaminoglycans, hyaluronic acid, cellulose derivatives, hydroxypropylmethylcellulose (HPMC), dextran, and any combination thereof.

33. The pharmaceutical composition of any one of claims 26-32, wherein the disaccharide is trehalose.

34. The pharmaceutical composition of any one of claims 26-33, wherein the cyclic oligosaccharide is beta-cyclodextrin.

35. The method of any preceding claim, wherein the composition further comprises a pH modifier, a pH buffer, a tonicity modifier, a stabilizer, an emulsifier, a preservative, a surfactant, a bulking agent, a flavorant, or any combination thereof.

36. The method of any preceding claim, wherein the composition further comprises a mucoadhesive.

37. The method of claim 36, wherein the mucoadhesive is selected from cellulose derivatives, polyacrylates, a starch, chitosan, glycosaminoglycans, hyaluronic acid, and any combination thereof.

38. The method of claim 36 or 37, wherein the weight percent of the mucoadhesive in the composition is about 0.1% to about 10% by weight.

39. The method of any preceding claim, wherein the MPLA compound is administered within 14 days of onset of symptoms of allergic rhinitis or chronic nasal congestion.

40. The method of claim 39, wherein the MPLA compound is administered within 5 days of allergic rhinitis or chronic nasal congestion.

41. The method of claim 39, wherein the MPLA compound is administered within 12 to 72 hours of onset of symptoms allergic rhinitis or chronic nasal congestion.

42. The method of any one of claims 1 to 41, wherein the MPLA compound is administered to the subject within 14 days of known or suspected exposure of the subject to an allergen.

43. The method of any one of claims 1 to 42, wherein the MPLA compound is administered within 5 days of known or suspected exposure of the subject to an allergen.

44. The method of any one of claims 1 to 42, wherein the MPLA compound is administered within 12 to 72 hours of known or suspected exposure of the subject to an allergen.

45. The method of any preceding claim, wherein the MPLA compound is administered about 1 to 72 hours prior to exposure of the subject to an allergen.

46. The method of any preceding claim, wherein the composition is administered to the subject once every 12 hours.

47. The method of any one of claims 1 to 45, wherein the composition is administered to the subject once every 24 hours.

48. The method of any one of claims 1 to 45, wherein the composition is administered to the subject once every 48 hours.

49. The method of any one of claims 1 to 45, wherein the composition is administered to the subject once every 72 hours.

50. The method of any preceding claim, wherein the compound is administered for a course of 1 week to 4 weeks.

51. The method of any preceding claim, wherein the composition is administered to the subject until the subject no longer experiences symptoms or is no longer exposed to the allergen.

52. The method of claim 51, wherein the MPLA compound is phosphorylated hexaacyl disaccharide (PHAD), PHAD-504, 3D-PHAD, 3D-(6-acyl) PHAD, or any combination thereof.

53. The method of claim 52, wherein the MPLA compound is PHAD.

54. The method of any preceding claim, wherein the composition is substantially free of a surfactant.

55. The method of any preceding claim, wherein the composition is substantially free of a phospholipid.

56. The method of any preceding claim, wherein the composition is substantially free of a salt, a buffer, or both.

57. The method of any preceding claim, further comprising concurrently administering at least one additional therapy.

58. The method of claim 57, wherein the at least one additional therapy comprises an antihistamines, a decongestant, a steroidal spray, or a combination thereof.

59. The method of claim 48, wherein the at least one additional therapy comprises diphenhydramine, loratadine, fexofenadine, cetirizine, brompheniramine, desloratadine, azelastine nasal, pseudophedrine, phenylephrine, oxymetazoline, and combinations thereof.
